(19) United States
(12) Patent Application Publication
(10) Pub. No.: US 2005/0010305 A1
(43) Pub. Date: Jan. 13, 2005

(54) Novel bone graft composite

(76) Inventor: Francis Y. Lee, Fort Lee, NJ (US)

Correspondence Address: LESLIE GLADSTONE RESTAINO;BROWN RAYSMAN MILLSTEIN & STEINER LLP, 55 MADISON AVENUE, 4TH FLOOR, MORRISTOWN, NJ, 07960, US

(21) Appl. No.: 10/766,084

(22) Filed: Jan. 28, 2004

Related U.S. Application Data
(60) Provisional application No. 60/443,525, filed on Jan. 28, 2003.

(51) Int. Cl. A61F 2/28

(52) U.S. Cl. 623/23.51

(57) ABSTRACT

A bone graft composite comprising a bisphosphonate is provided which is suitable for treating bone destruction. The bisphosphonate provides bone graft protection.

### RELATED APPLICATIONS

This application claims the benefit of U.S. Provisional patent application Ser. No. 60/443,525, filed on Jan. 28, 2003, and entitled “NOVEL BONE GRAFT COMPOSITE”, the contents of which are hereby incorporated by reference herein.

### FIELD OF THE INVENTION

The invention disclosed herein generally relates to a novel bone graft composite. The invention also relates to therapeutic applications using such bone graft composite.

### BACKGROUND OF THE INVENTION

Benign bone tumors such as giant cell tumor (GCT), unicameral bone cyst (UBC) an aneurismal bone cyst (ABC) are characterized by bone destruction that is mediated by osteoclasts. The consequences of benign bone tumors include bone destruction and pathologic fractures. Most of bone tumors occur near the joint and pathologic fractures can further destroy the joints. Affected patients often require joint replacement surgeries. These bone tumors often are recurring and the typical example is GCT that also recurs in 30% to 40% of the cases. Other examples include UBC and ABC that occur in growing children. Recurrence is greater than 30% to 50% after a single treatment such as injection of bone marrow or bone grafting. UBC and ABC cause recurrent fractures, deformity and growth disturbances.

Most of the currently available treatments consist of mechanical removal of a tumor, bone grafting using allografts and internal fixation to restore the structural integrity of bone. Bone grafts are often dissolved by reactivation of tumor cells and lose the goal of structural restoration. The drawbacks associated with surgical treatment include postoperative morbidity, and recurrence and resorption (or loss) of bone grafts. The recurrent lesions are characterized by resorption of the transplanted bone.

Understanding the fundamental pathophysiology of bone destruction is the first step in designing novel therapeutic strategies. In the past, bone destruction was thought to be the consequence of destructive effects of tumor cells. It has been suggested that tumor cells attract and activate monocytes, which in turn fuse to form osteoclasts.

Mediation of osteoclast activation involves specific gene activation such as receptor activator of nuclear factor κβ ligand, (RANKL). RANKL stimulates the formation of multinucleated osteoclasts from monocytic precursors. Bone tumor cells attract immune cells and express RANKL to destroy the bone. Osteoclasts, which can melt the bone, are abundant in primary and metastatic bone tumors. After discovery of major signaling molecules such as osteoprotegerin (OPG; Osteoclastogenesis Inhibiting Factor) or receptor activator of nuclear factor kappa κβ: (RANKL; OPG ligand; TRANCE; Osteoclastogenesis Factor), molecular mechanisms by which bone is destroyed has been better elucidated.

Bisphosphonates have a history of successful clinical use for the treatment and prevention of pathological bone destruction of various origins, such as osteolytic bone diseases due to malignancy, Paget's disease, osteogenesis imperfecta, hypercalcemia caused by malignancy, and tumor metastases in bone including multiple myeloma. There are a number of bisphosphonates currently available that have been approved for such clinical use. Such bisphosphonates include but are not limited to Alendronate, Etidronate, Pamidronate, Zolendronate, Risedronate and Tiludronate. The mode of delivery of such bisphosphonate may be oral or intravenous. Limited absorption from the gastrointestinal tract and fast appearance of bisphosphonate following intravenous administration are all characteristics of known bisphosphonates. Bisphosphonates are rapidly cleared from plasma. The half-life in bone however, is very long, partially as long as the half-life of the bone in which they are replaced.

Bisphosphonates are analogues of the physiologically occurring inorganic pyrophosphates. Selective action of the bisphosphonate on bone is based on the binding of the bisphosphonate moiety to the bone mineral. However, the molecular mode of action remains unclear and may differ from compound to compound. At the tissue level, all bisphosphonates inhibit bone destruction and lead to an increase in bone mineral density by decreasing bone resorption and bone turnover. At the cellular level, the ultimate target of bisphosphonate action is the osteoclast. It is likely that bisphosphonates are internalized by osteoclasts and interfere with specific biochemical processes and induce apoptosis. Recent studies show that bisphosphonates can be classified into at least 2 groups with different modes of action. Bisphosphonates that closely resemble pyrophosphate (such as clodronate and etidronate) can be metabolically incorporated into nonhydrolysable analogues of ATP that may inhibit ATP-dependent intracellular enzymes. The more potent, nitrogen containing bisphosphonates (such as zolendronate, pamidronate, alendronate, risedronate, and ibandronate) are not metabolized in this way but can inhibit enzymes of the mevalonate pathway, thereby preventing the biosynthesis of isoprenoid compounds that are essential for the posttranslational modification of small GTPases. The inhibition of protein prenylation and the disruption of the function of these key regulatory proteins explain the loss of osteoclast activity and induction of apoptosis. The P—C—P bond of the bisphosphonates is completely resistant to enzymatic hydrolysis (Fleisch H., Bisphosphonates in Bone Disease, Academic Press, 2002, Ch. 22, pp. 30-33).

The present invention makes use of the effect of the bisphosphonates in inhibiting the activity of osteoclasts and preventing bone resorption in the design of a novel bone graft composite. Such bone graft composite finds applicability in treating or decreasing tumor recurrence, decreasing osteoclast activity and formation, elimination of osteoclasts and restoration of bone loss.

### SUMMARY OF THE INVENTION

The present invention provides for bone graft composite comprising a bisphosphonate, a bone graft and a carrier material. In a preferred embodiment, such bisphosphonate is Pamidronate.

The present invention also provides for a method of treating a patient suffering from bone destruction by inserting a bone graft composite at the site of such bone destruction.

Additional aspects of the present invention will be apparent in view of the description that follows.

### BRIEF DESCRIPTION OF THE FIGURES

FIG. 1 is a diagram illustrating the therapeutic rationale using the bone graft composite of the invention.

FIG. 2 is a diagram showing that Pamidronate induces apoptosis of giant cell tumors in a dose dependent manner. The control picture shows plumpy, polyhedral cytoplasm. Addition of 100 and 200 μM of Pamidronate induces cell death. Annexin V staining indicates apoptosis of tumor cells.

FIG. 3 is a diagram showing an apoptosis (*) and necrosis assay using Annexin V and propium iodide. The apoptotic population increases with a higher dose of Pamidronate.

FIG. 4 shows the effect of the bone graft composite of the invention on the giant cell tumor and unicameral bone cyst.

### DETAILED DESCRIPTION OF THE INVENTION

The definitions below serve to provide a clear and consistent understanding of the specification and claims, including the scope to be given such terms.

By the term “bone” is intended for the purposes of the present invention, bone recovered from any source including animal and human, for example, human bone recovered for the production of allografts, and animal bone recovered for the production of xenografts, such allografts and xenografts suitable for implantation into a human.

By the term “bisphosphonate” is intended for the purposes of the present invention to include without limitation a bisphosphonate such as Pamidronate, Alendronate, Etidronate, Zolendronate, Risendronate and Tiludronate.

By the term “bone graft material” is intended for the purposes of the present invention to include hydroxyapatite, tricalcium phosphate, synthetic material such as autogenous bone graft, gel foam, bone cement or any other calcium containing material.

By the term “carrier material” is intended for the purposes of the present invention to include bone chips or any other calcium containing material.

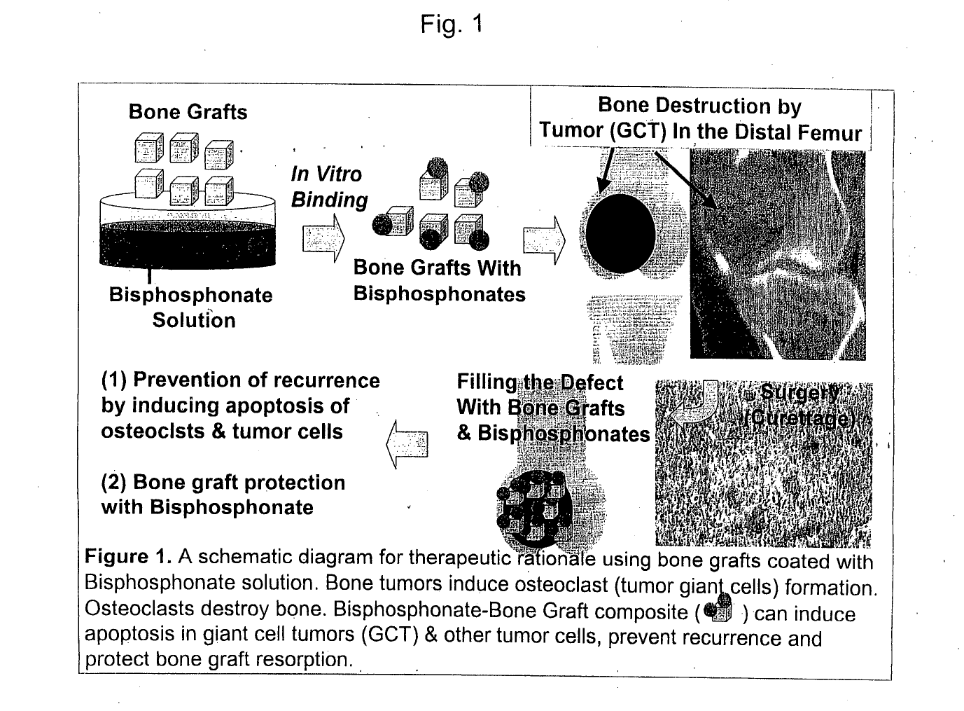

A therapeutic rationale has been developed using bisphosphonate in a bone graft composite. In one embodiment, the bone graft composite includes a bisphosphonate in a carrier material and a bone graft material. In another embodiment, the bone graft composite includes a bisphosphonate and a bone graft material. The bone graft composite can be formed by mixing with a bisphosphonate solution bone graft material. A carrier material may also be included and mixed in. When the carrier material is not included, the bone graft material serves the dual purpose of bone graft and carrier materials. As shown in FIG. 1, prior to implantation of the bone graft composite, a surgical procedure is performed to remove the tumor cells from the anatomic site of tumor. The choice of surgical procedure is dictated by the site of primary pathology and by the physical size of the bone graft composite. Following removal of the tumor cell, the bone graft composite is implanted. The bone graft composite prevents recurrence by inducing apoptosis of osteoclasts and tumor cells. In addition, the composite provides protection with bisphosphonate against bone graft resorption and tumor recurrence.

To study the effect of bisphosphonate, the inventor has identified tumor stromal cells derived monocyte chemotactic factor (SDF-1) in the giant cell tumor of bone as a model to provide insight into the molecular interaction between tumor cells and host immune system that generate osteoclasts. These tumor cells undergo apoptosis in response to an antiresorptive agent in vitro. The cells were grown from the giant cell tumors. The giant cell tumor is comprised of neoplastic stromal cells, monocytes and osteoclast-like multinucleated cells. The neoplastic stromal cells express RANKL, which stimulates monocytes to form multinucleated giant cells. This is generally observed after several passages of cell lines, suggesting autocrine effect of neoplastic cell-monocyte interaction. In order to identify monocyte-attracting factors, RNAs from the tumor tissue and cell lines were hybridized with stromal cell derived factor (SDF-1). The tumor expressed SDF-1 that may mediate the molecular interaction between neoplastic tumor cells and monocytes. Specific therapeutic regimens can be designed to block the osteoclastogenesis from monocytes, to inhibit osteoclasts and to inhibit or decrease monocyte chemoattractive factors.

Several factors have an effect on the selection of a bisphosphonate. These factors include the level of osteoclasts inhibition by the particular bisphosphonate both in vitro and in vivo. Other factors include the tolerance of such bisphosphonate by the bone tissue, and the tolerance of such bisphosphonate by a patient.

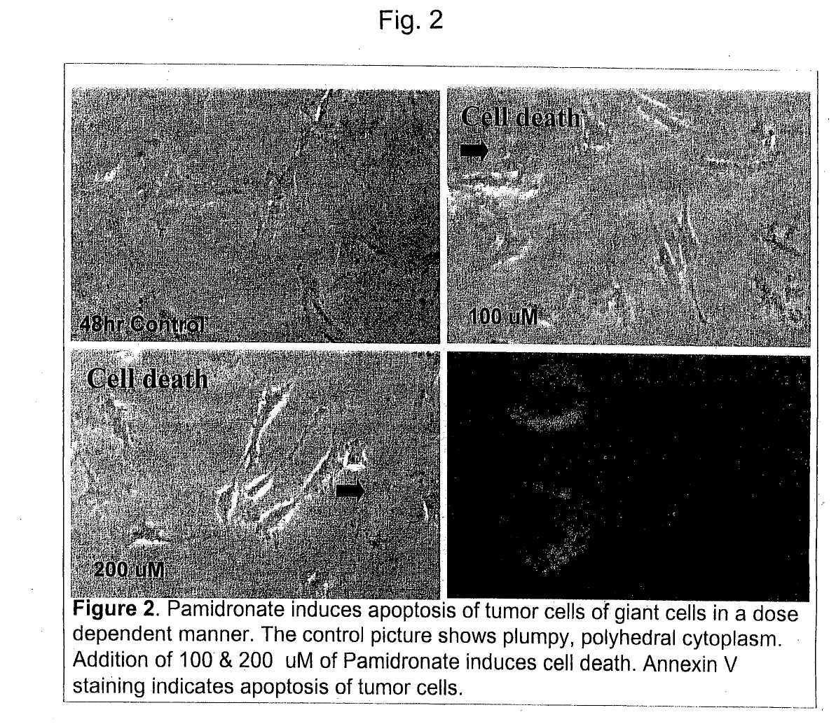

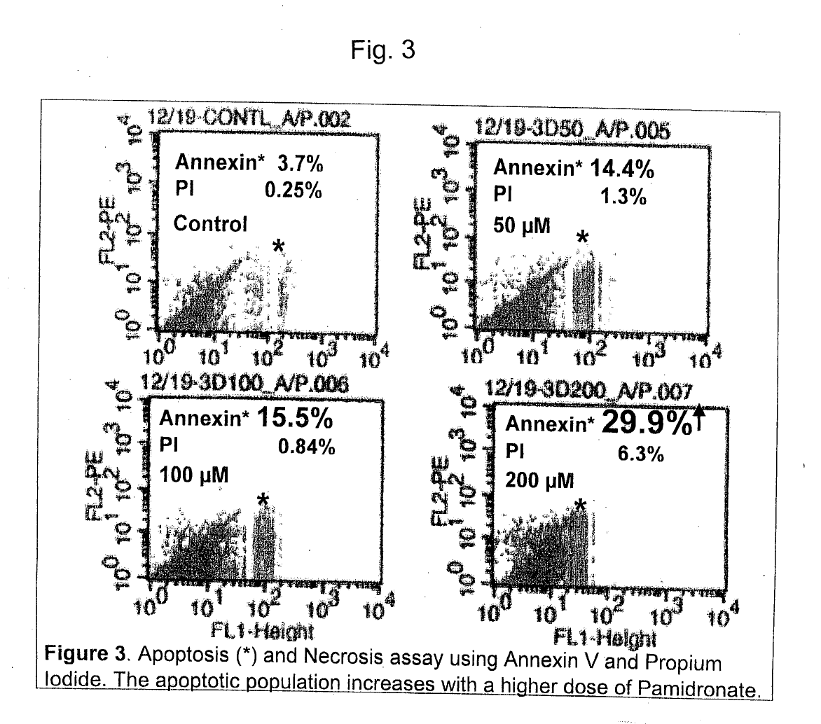

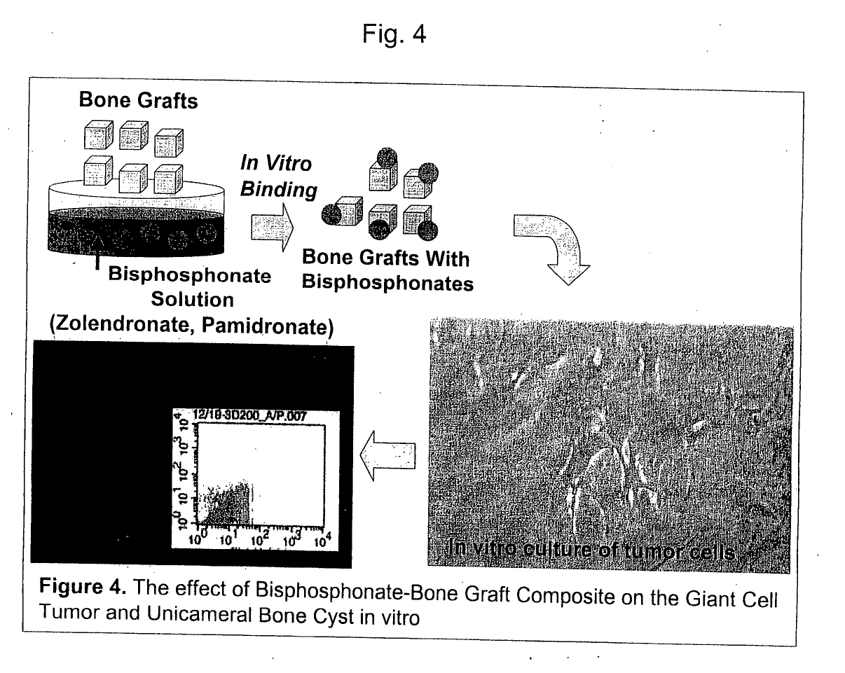

For the purpose of demonstrating the effect of bisphosphonate, in vitro assays were performed using Pamidornate. Pamidronate is well tolerated by patients with metastatic bone cancers and osteoporosis. FIG. 2 shows the shrunken and irregular morphology of tumor cells after treatment with Pamidronate, and shows that Pamidronate at concentrations of 50 μm, 100 μm and 200 μm induces apoptosis of tumor cells of giant cells in a dose-dependent manner. FIG. 3 shows apoptosis and necrosis assay using Annexin V and propium iodide. The apoptotic population increases with a higher dose of Pamidronate. FIG. 4 shows the effect of the bone graft composite of the invention in the giant cell tumor and unicameral bone cyst in vitro. The tumor cells are shown to regress when brought into contact with the bone graft composite of the invention. It will be appreciated by one skilled in the art that the results in FIGS. 2, 3 and 4 are in no way limited to a particular bisphosphonate.

The present bone graft composite is useful for implantation in patients suffering from defects caused by pathological bone destruction of various origins such as osteolytic bone disease. Those of ordinary skill in the art to which the present invention pertains can readily select and employ a particular bone graft composite without undue experimentation. Factors to be considered in such selection and employment include: the type and size of graft bone, its anatomic site of fusion, and the age of the patient. Graft selection and surgical techniques are factors that can be readily selected, optimized and employed by those of ordinary skill in the art without undue experimentation and are discussed in various references (e.g. Campanaci, M., Baldini, N., Boriani, S., Sudanese, A., Giant Cell Tumors of Bone, J. Bone Joint Surg. (Am), 1987, 69-106-114).

While the foregoing invention has been described in some detail for purposes of clarity and understanding, it will be appreciated by one skilled in the art, from a reading of the disclosure, that various changes in form and detail can be made without departing from the true scope of the invention in the appended claims.

1. A bone graft composite comprising a bisphosphonate.

2. The composite of claim 1 wherein said bisphosphonate is Pamidronate.

3. The composite of claim 1 further comprising a bone graft.

4. The composite of claim 1 further comprising a carrier material.

5. A bone graft composite comprising a bisphosphonate, a bone graft and a carrier material.

6. The composite of claim 5 wherein said bisphosphonate is Pamidronate.

7. A method for treating a patient suffering from bone destruction, the method comprising the step of inserting at the site of bone destruction a bone graft composite, wherein said bone graft composite replaces diseased bone at the site of bone destruction.

8. The method of claim 7 wherein the insertion step is preceded by surgical removal of the diseased bone.

9. The method of claim 7 wherein said bone graft composite comprises a bisphosphonate.

10. The method of claim 9 wherein said bisphosphonate is Pamidronate.
